(19) United States
(12) Patent Application Publication
(10) Pub. No.: US 2014/0242654 A1
(43) Pub. Date: Aug. 28, 2014

(54) Butyrate Producing Clostridium species, Clostridium pharus

(71) Applicant: COSKATA, INC., Warrenville, IL (US)

(72) Inventor: William E. Levinson, Naperville, IL (US)

(73) Assignee: COSKATA, INC., Warrenville, IL (US)

(21) Appl. No.: 13/777,150

(22) Filed: Feb. 26, 2013

(51) Int. Cl. C12N 1/20

(52) U.S. Cl. CPC C12N 1/20
USPC 435/141; 435/252.7

(57) ABSTRACT

A clostridia bacterial species Clostridium pharus, is provided. Under anaerobic conditions Clostridium pharus can convert ethanol and/or acetate to butyric acid.

### FIELD OF THE INVENTION

The field of the invention relates to a bacterial species that is capable of producing butyrate from ethanol and acetate. In particular, the invention provides a Clostridium species, Clostridium pharus, having the identifying characteristics of ATCC No. PTA-13419 and a method of synthesizing butyrate from ethanol and acetate using this Clostridium species.

### BACKGROUND OF THE INVENTION

Currently, the only anaerobic, clostridial, bacterium known to grow on ethanol and acetate as the sole energy sources is C. kluyveri with the fermentation products of this metabolism being butyrate and hexanoate. Seedorf, Fricke et al. (2008).

Butyric acid and its conjugate base, butyrate ion (or butyrate salts) are widely used industrial compounds with applications in the chemical, food, and pharmaceutical industries. Its main use is as a component in the manufacture of cellulose acetate butyrate (CAB) plastics and butyric acid-esters, which are used as flavor additives in the food industry (Zhang, Yang et al. 2009, Dwidar, Park et al. 2012). Ethyl-butyrate and butyl-butyrate are butyrate derivatives that may be used as fuels (Dwidar, Park et al. 2012). In addition, butyrate may be converted to n-butanol, which is an important industrial chemical with a wide range of applications. It can be used as a motor fuel particularly in combination with gasoline to which it can be added in all proportions. Currently the world production of n-Butanol is 3.5 million tons/yr (7.7 billion lb/yr). Furthermore, conversion of alcohols to long chain linear hydrocarbons that would be suitable for jet fuel use are being developed and demonstrated, which could further increase the demand for n-Butanol (Cobalt Technologies) (2012).

While butyric acid is currently produced industrially by chemical synthesis using petrochemical-based feedstocks, its production from renewable resources has been investigated. It may be produced by fermentation with organisms belonging to several different genera of bacteria, however a number of Clostridium species have been the most widely-studied for this purpose (Zhang, Yang et al. 2009, Dwidar, Park et al. 2012). Included in this group are strains of C. butyricum, C. tyrobutyricum and C. thermobutyricum (Zhang, Yang et al. 2009, Dwidar, Park et al. 2012). While having different substrate specificities, all of these organisms ferment sugars or sugar polymers to produce butyric acid.

The limitations for carbohydrate feedstocks for use in butyric acid production are well known and some are fundamental. Starch and sugars from agricultural crops run into competing issues of food vs. energy/chemical production as well as the cost of the feedstocks and their availability. For lignocellulosic feedstocks such as woody biomass, grasses etc. the cost and yield from pretreatment and hydrolysis processes are very limiting. For example, typical woody biomass contains 50% cellulose while the remainder consists of hemicelluloses, lignin and other fractions. The chemical energy content of the fermentable fractions is often less than 50% of that of the feedstock, putting fundamental limitations on product yield, therefore alternatives to carbohydrate fermentations are required.

Several microorganisms are able to use one-carbon compounds as a carbon source and some even as an energy source. Gasification technology converts all the components of the feedstock primarily to a mixture of CO, H₂, CO₂ and some residual CH₄ and the synthesis gas produced is a common substrate for supplying the one carbon compounds such as CO and CO₂ (as well as hydrogen) used in syngas fermentations. Furthermore, a wide range of feedstocks, both renewable, such as woody biomass, agricultural residues, or municipal wastes, and non-renewable, such as natural gas, can be gasified to produce these primary components.

When the syngas source is biomass, gasification of the whole biomass may be performed without the need of pretreatment to unlock certain fractions. Typically, this gasification yields 75 to 80% cold gas efficiency, i.e. 75 to 80% of the chemical energy of the feedstock is available for further chemical or biological conversion to target products. The rest of the energy is available as heat that can be used to generate steam to provide some or all of the process energy required. Natural gas can be economically reformed to syngas with a wide variety of technologies using steam, oxygen, air or combinations thereof. This syngas has very good cold gas efficiency of approximately 85% to produce CO, H2 and CO2 with a wide range of target compositions.

Hence, syngas is an economical feedstock that can be derived from a wide range of raw materials both renewable and non-renewable. Thus, conversion of syngas to butyrate (via ethanol and acetate) with high yield and concentrations could lead to economical production of this important chemical.

An efficient conversion of syngas takes place when fermenting it to ethanol and acetate. Acetogens are a group of anaerobic bacteria able to convert syngas components, like CO, CO₂ and H₂ to acetate via the reductive acetyl-CoA or the Wood-Ljungdahl pathway. Fermentation of syngas to ethanol and acetate offers several advantages such as high specificity of the biocatalysts, lower energy costs (because of low pressure and low temperature bioconversion conditions), greater resistance to biocatalyst poisoning and nearly no constraint for a preset H₂ to CO ratio (Klasson, Ackerson et al. 1992, Bredwell, Srivastava et al. 1999). Several anaerobic bacteria have been isolated that have the ability to ferment syngas to ethanol, acetic acid and other useful end products.

Despite the knowledge in the art regarding the use of microorganisms in the production of biofuels and chemicals, there remains an ongoing need to discover and/or develop additional microorganisms that are capable of producing useful products from fermentation processes. In particular, it would be advantageous to discover new organisms capable of fermenting syngas or utilizing the products of syngas fermentation by acetogens (primarily ethanol and acetate) to produce value-added compounds.

### SUMMARY OF INVENTION

The present invention is directed to an isolated biologically pure culture of a newly discovered butyrogenic Clostridium species, Clostridium pharus having the genotypic characteristics of ATCC PTA-13419. This species is a new Clostridial species with distinguishing phenotypic and genetic characteristics and fermentation productions from other known Clostridia species. In particular aspects of the invention the isolated biologically pure culture of the microorganism Clostridium pharus, ATCC No. PTA-13419 has the ability, under anaerobic conditions, to produce butyrate from energy sources selected from the group consisting of ethanol, acetate and propanol, or combinations thereof. In particular embodiments the energy sources are provided by fermentation of syngas.

In other aspects of the invention the isolated biologically pure culture of the microorganism Clostridium pharus comprises the 16S rDNA sequence as set forth in SEQ ID NO. 1.

In yet other aspects of the invention methods of producing butyrate comprising the steps of contacting the Clostridium pharus with energy sources selected from the group consisting of ethanol and acetate or combinations thereof and propanol and acetate and combinations thereof under conditions which allow the Clostridium pharus to convert the energy sources to butyrate are provided.

Specific preferred embodiments of the invention will become evident from the following more detailed description of certain preferred embodiments and the claims.

### BRIEF DESCRIPTION OF DRAWINGS

FIG. 1 is a phase-contrast light micrograph of Clostridium pharus cells during late log-phase, 1,000× magnification.

FIG. 2 is a scanning electron micrograph of Clostridium pharus cells harvested during late log phase growth.

FIG. 3 is the 16s rDNA sequence of Clostridium pharus.

FIG. 4 is a table of the top Blast results of the Clostridium pharus 16S rDNA sequence against the NCBI nucleotide sequence database (excluding uncultured and environmental samples). The actual percentage of nucleotide identities of the Clostridium pharus query to C. kluyveri match is 97.7%.

FIG. 5-FIG. 5(a) illustrates a Parsimony tree and FIG. 5(b) illustrates a Pearson UPGMA similarity matrix scores both for the analysis of amplicons generated by REP-PCR for three strains of Clostridium pharus, C. kluyveri and the homoacetogen, C. ljungdahlii.

FIG. 6 illustrates a Parsimony tree and Pearson UPGMA similarity matrix scores for the analysis of amplicons generated by BOX-PCR for three strains of Clostridium pharus, C. kluyveri and the homoacetogen, C. ljungdahlii.

FIG. 7 illustrates a Parsimony tree and Pearson UPGMA similarity matrix scores for the analysis of amplicons generated by ERIC-PCR for three strains of Clostridium pharus and C. kluyveri.

FIG. 8 illustrates a Parsimony tree and Pearson UPGMA similarity matrix scores for the analysis of amplicons generated by HsdR-PCR for three strains of Clostridium pharus, C. kluyveri and the homoacetogen, C. ljungdahlii.

FIG. 9 illustrates the temperature growth optimums of Clostridium pharus and C. kluyveri at 15 hours of incubation based on optical density. The graph demonstrates the OD 600 nm-Balch tubes vs. temperature. The table is a qualitative representation of growth vs. incubation temperature.

FIG. 10 illustrates the medium pH growth optimums of Clostridium pharus and C. kluyveri at 48 hours of incubation based on optical density. Graph data demonstrates the average of duplicates. The table is a qualitative representation of growth vs. medium pH.

FIG. 11 represents the positive reactions for Clostridium pharus and C. kluyveri derived from a Biolog plate test.

FIG. 12 represents the positive reactions for Clostridium pharus and C. kluyveri derived from an API 20A test strip.

FIG. 13 represents the positive reactions for Clostridium pharus and C. kluyveri derived from an API Rapid ID 32A test strip.

FIG. 14 is a table indicating substrate combinations of alcohols and acids that can be used for growth by Clostridium pharus and C. kluyveri.

FIG. 15 is a graph displaying the product profiles of Clostridium pharus and C. kluyveri after approximately 24-hours growth on ethanol and acetate.

FIG. 16 is a graph displaying the fatty acid methyl-ester profiles of cultures of C. pharus and C. kluyveri.

### DETAILED DESCRIPTION OF EMBODIMENTS

The bacterium of the present invention is a butyrogenic Clostridium species which displays the characteristics represented by ATCC No. PTA-13419, herein referred to as “Clostridium pharus”. The phylogenetic, morphological, and biochemical properties of Clostridium pharus have been analyzed and are described in the Examples section below. Clostridium pharus possesses unique characteristics that confirm it is a novel species of this genus. The data included in the examples shows that this bacterium is a new representative of the Clostridium genus. Butyrogens, as referred to hereafter, is any microorganism capable of converting syngas intermediates, such as ethanol and acetate, or acetate, ethanol and some hydrogen to primarily n-butyrate.

The full 16S rRNA identity of Clostridium pharus compared to C. kluyveri is 97.7%. While there is debate in the field of what constitutes genomic uniqueness of a novel isolate, ≦98.7-99% difference between 16S rRNA gene sequence has been generally accepted in the field as constituting a novel species. (Stackebrandt and Ebers 2006, Janda and Abbott 2007). Scores of >99% are not clear, and require the use of methods with higher resolution such as DNA-DNA reassociation, or DNA fingerprinting (Konstantinidis and Tiedje 2005, Janda and Abbott 2007).

Clostridium pharus has the ability, under anaerobic conditions, to produce butyrate from the fermentation of ethanol and acetate. Butyrate is the primary product of this fermentation. No hexanoate is produced by this organism.

Butyric acid and butyrate are an acid/base pair and, as such, the relative concentrations of the different forms (acid or butyrate ion) produced using the methods of the present invention will be dependent on the pH of the fermentation.

Any source of ethanol and acetate may be utilized by the Clostridium of the present invention. For example, preferred sources of these substrates include ethanol and acetate produced by the fermentation of syngas by homoacetogenic bacteria. Alternatively, the growth substrates may be produced from other sources for use in the methods of the invention. Those of skill in the art will recognize that any source of substrates may be used in the practice of the present invention, so long as it is possible to provide Clostridium pharus with sufficient quantities of the substrates under conditions suitable for the bacterium to carry out the fermentation reactions.

The Clostridium pharus of the present invention must be cultured under anaerobic conditions. “Anaerobic conditions” means the level of oxygen (O₂) is below 0.5 parts per million in the gas phase.

In general, the optimized butyrate production media for culturing the butyrogen of this invention is a liquid medium that is chemically-defined. However, those of skill in the art will recognize that alternative media can be utilized. Further, various media supplements may be added for any of several purposes, e.g. buffering agents, metals, vitamins, and salts. In particular, those of skill in the art are familiar with such techniques as nutrient manipulation and physiological adaptation, which result in increased or optimized yields of a bioproduct and all such optimized procedures using Clostridium pharus are intended to be encompassed by the present invention.

In particular, Clostridium pharus may be cultured using the Balch technique (Balch and Wolfe 1976, Balch, Fox et al. 1979), which are incorporated herein by reference. This entails the aid of an anaerobic chamber for preparing culture materials and a gas exchange manifold to establish whatever gas phase is desired for culture in sealed tubes or vessels. Methods to enhance butyrate production include optimization of key medium components (such as iron, phosphate and vitamins), control of culture pH, random mutagenesis of the bacterium followed by clonal screening, or genetic engineering of the bacterium.

The metabolism of the growth substrate supplied to the fermentation of Clostridium pharus can be carried out in any of several types of apparatuses that are known to those of skill in the art, with or without additional modifications, or in other styles of fermentation equipment that are currently under development. Examples include but are not limited to bubble column reactors, two stage bioreactors, trickle bed reactors, membrane reactors, packed bed reactors containing immobilized cells, etc. The chief requirements of such an apparatus include:
(1) Axenicity;
(2) Anaerobic conditions;
(3) Suitable conditions for maintenance of temperature, pressure, and pH;
(4) Sufficient quantities of substrates are supplied to the culture;
(5) The end products of the fermentation can be readily recovered from the bacterial broth.

The Examples which follow are illustrative of specific embodiments of the invention, and various uses thereof. They are set forth for explanatory purposes only, and are not to be taken as limiting the invention.

### Example 1

### Isolation of Clostridium pharus

Clostridium pharus was obtained from an ethanol producing anaerobic syngas fermentation that began to produce butyrate and butanol. Samples from the fermentation were inoculated to an ethanol and acetate containing growth medium for the production of single colony isolates by use of a roll-tube method. Isolates obtained by this method were grown in liquid medium with ethanol and acetate as substrates prior to being subjected to a second round of roll-tube isolation. The isolates obtained were screened for product profile and examined by microscopy to verify purity. Pure cultures with the desired product profile were submitted for 16s rDNA sequencing. The bacterium obtained, Clostridium pharus, was deposited with the American Type Culture Collection as strain ATCC No. PTA-13419 on Jan. 8, 2013.

A butyrogen growth medium, described below in Tables A-G, was used for isolation and maintenance of the organism. Roll-tubes for isolation contained 2% agar. The headspace for growth was 20% CO₂/balance N₂ at approximately 15 psi. Cultures used in the following examples were grown at a temperature of 37° C. on an orbital shaker at 100 rpm unless otherwise noted. The culture medium was prepared using the strict anaerobic technique described by Balch and Wolfe (1976). After sterilization, post-autoclave additions were made to the sealed bottles from sterile, anoxic, stock solutions as detailed in Table G. After inoculation, the headspace was exchanged to the mixture detailed above. Standard inoculum size was 10% (v/v).

TABLE A
Mineral stock solution

| Item | Component | Amount, g/L |
|---|---|---|
| 1 | NaCl | 80 |
| 2 | NH₄Cl | 100 |
| 3 | KCl | 10 |
| 4 | KH₂PO₄ | 10 |
| 5 | MgSO₄•7H₂O | 20 |
| 6 | CaCl₂•2H₂O | 4 |

TABLE B
Trace metals stock solution.

| Item | Component | Amount, g/L |
|---|---|---|
| 1 | Hydrochloric acid, 12.1N | 2.360 |
| 2 | MnSO₄•H₂O | 1.0 |
| 3 | Fe(NH₄)₂(SO₄)₂•6H₂O | 0.8 |
| 4 | CoCl₂•6H₂O | 0.2 |
| 5 | ZnSO₄•7H₂O | 1.0 |
| 6 | NiCl₂•6H₂O | 0.2 |
| 7 | Na₂MoO₄•2H₂O | 0.02 |
| 8 | Na₂SeO₄ | 0.1 |
| 9 | Na₂WO₄ | 0.2 |

TABLE C
Vitamin stock solution.

| Item | Component | Amount, mg/L |
|---|---|---|
| 1 | Pyridoxine, HCl | 10 |
| 2 | Thiamine, HCl | 5 |
| 3 | Riboflavin | 5 |
| 4 | Calcium pantothenate | 5 |
| 5 | Thioctic Acid | 5 |
| 6 | p-Aminobenzoic Acid | 5 |
| 7 | Nicotinic acid | 5 |
| 8 | Vitamin B12 | 5 |
| 9 | Mercaptoethanesulfonic acid | 5 |
| 10 | Biotin | 2 |
| 11 | Folic Acid | 2 |

TABLE D
Reducing agent stock solution.

| Item | Component | Amount, g/L |
|---|---|---|
| 1 | Cysteine (free base) | 40 |
| 2 | Na₂S•2H₂O | 40 |

TABLE E
Supplementary Trace Metals (Sterile; Anoxic).

| Item | Component | Amount, g/L |
|---|---|---|
| 1 | CuCl₂•2H₂O | 0.063 |
| 2 | H₃BO₃ | 0.05 |
| 3 | AlK(SO₄)₂•12H₂O | 0.019 |

pH adjusted to 2.0 with HCl prior to metals addition.

TABLE F
Sterile, anoxic stock solutions for post-autoclave addition.

| Component | Concentration |
|---|---|
| KHCO₃ | 1M |
| Sodium Acetate | 2M |
| Ethanol | 50% |

TABLE G
Final Medium Composition.

| Item | Component | Amount | |
|---|---|---|---|
| 1 | Minerals stock solution | 25 | mL/L |
| 2 | Trace metals solution | 10 | mL/L |
| 3 | Vitamin stock solution | 10 | mL/L |
| 4 | Yeast extract | 0.5 | g/L |
| 5 | 2-(N-morpholino)ethanesulfonic acid buffer | 20 | g/L |
| | (MES) | | |
| 6 | 5N NaOH | Adjust pH to 5.8 | |
| 7 | 0.1% aqueous resazurin | 0.5 | mL/L |
| 8 | Reducing agent | 2.5 | mL/L |
| 9 | Supplementary Trace Metals | 10 | mL/L |
| 10 | KHCO₃ (1M) | 10 | mL/L |
| 11 | Sodium Acetate (2M) | 50 | mL/L |
| 12 | Ethanol (50%) | 20-40 | mL/L |

Items 9 through 12 were added to the medium bottles post-autoclave from sterile, anoxic stock solutions.

### Example 2

### Cellular Morphology and Growth

Cells were prepared for scanning electron microscopy (SEM) by filtration onto a polycarbonate membrane followed by fixation with 3% glutaraldehyde at 4° C. overnight. After fixing, the cells were dried with an ethanol rinse series (20, 40, 60, 80 100%) for 10 minutes each.

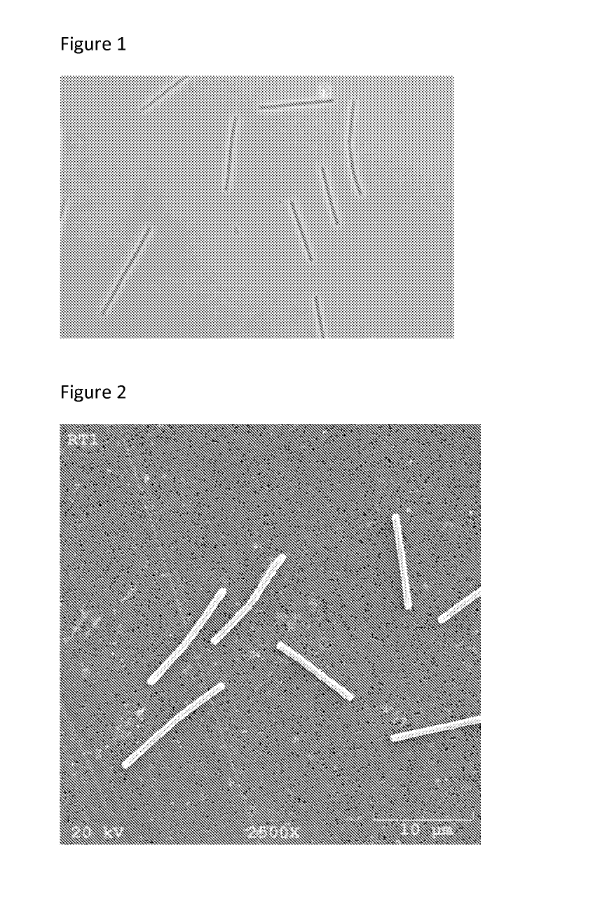

Cells of Clostridium pharus are large, straight rods; occurring singly, as diploids or occasionally as longer chains. (FIG. 1). Motility or spore formation was not observed. Cells observed by SEM (FIG. 2) were approximately 1 μm in diameter and ranged from approximately 8 to 13 μm in length for single cells. C. kluyveri cells examined by the same method were about the same diameter but ranged in length from about 3 to 9 μm.

### Example 3

### 16S rRNA Sequences

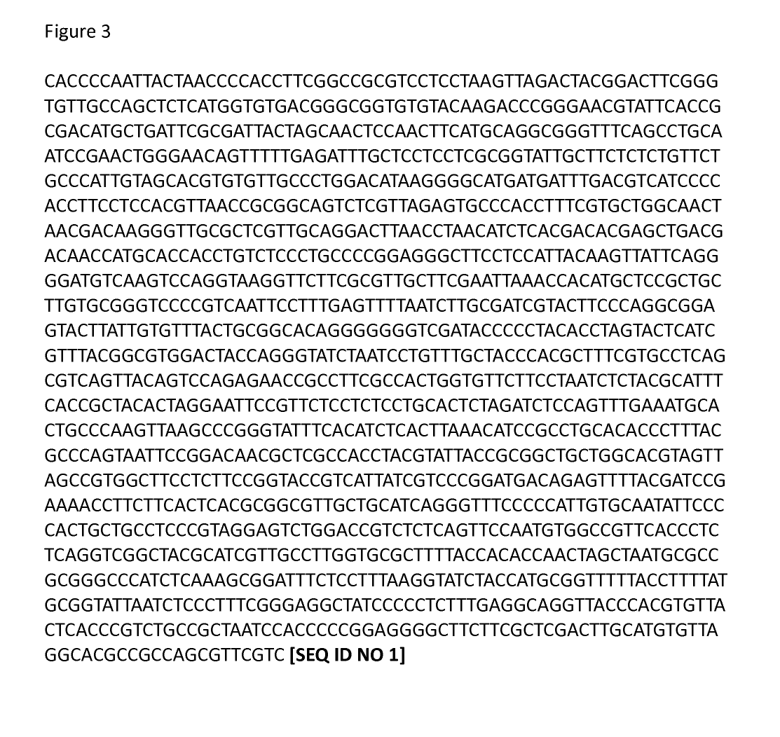

The sequence of the 16s rDNA gene was determined for four isolates of Clostridium pharus (isolated through the second round of roll-tube isolation) with the same product profile characteristics and cell morphologies. Genomic DNA preparations were made from liquid cultures and these were used as template for PCR reactions with the 27F/1492R 16s universal primer pair to amplify the 16s rDNA sequence. The product of the PCR reactions were purified with a PCR clean-up kit and the resulting amplicon was submitted to an outside lab for sequencing. Sequencing was performed with the same primers as amplification as well as with complementary primers binding to internal priming sites of the sequence. The four sequences returned for each isolate were assembled into a single contig. These contigs were then aligned against each other and found to be identical as shown by the consensus sequence (FIG. 3).

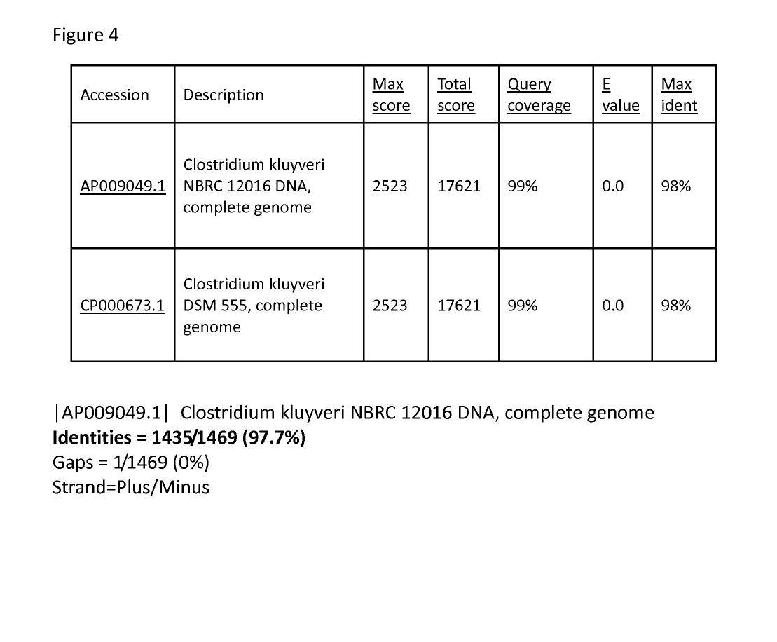

The consensus sequence was then submitted for a Blast search (Basic Local Alignment Search Tool) on the National Center for Biotechnology Information website (NCBI). The sequence was queried against the NCBI nucleotide database excluding uncultured/environmental sequences (FIG. 4). The two highest identity sequence matches from the search are presented in FIG. 4. Both sequence alignments returned were from type strains of C. kluyveri and the alignments were identical. Detailed identity information comparing the isolate sequence to the C. kluyveri sequence demonstrated that the sequence identity was 97.7% (1435 of 1469 nucleotides identical) (FIG. 4). This level of identity is below that of 98.7% proposed by Stackebrant and Ebers (Stackebrandt and Ebers 2006) as the cut-off for a novel species using 16S rRNA homology.

### Example 4

### PCR-Based DNA Fingerprinting

One method for comparing closely related organisms is DNA fingerprinting by REP-PCR. This method makes use of DNA primers complementary to naturally-occurring, highly conserved repetitive DNA sequences that are present in multiple copies in most bacteria. The length and concentration of the amplicons that result from PCR provides a highly specific DNA fingerprint that can differentiate closely related species, or may be used to establish organisms that are genetically identical (Tindall, Rossello-Mora et al. 2010).

Four REP-PCR type methods were used for DNA fingerprinting of clostrial genera closely related to Clostridium pharus based on 16s rRNA sequence, which included repetitive extragenic palindromic elements (REP-PCR), conserved repetitive DNA elements (BOX-PCR), entrobacterial repetitive PCR intergenic consensus sequences (ERIC-PCR) and PCR of the Type I restriction-modification enzyme R subunit (HsdR-PCR).

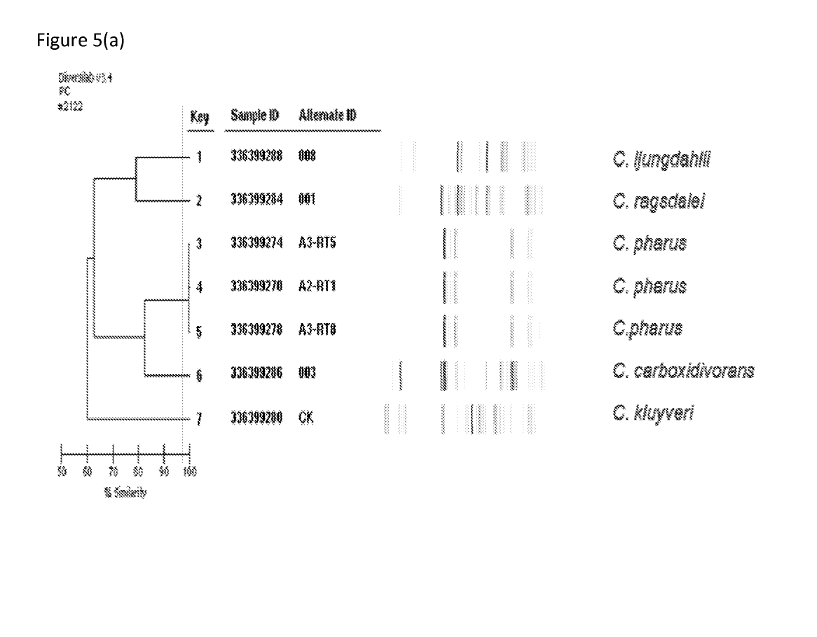

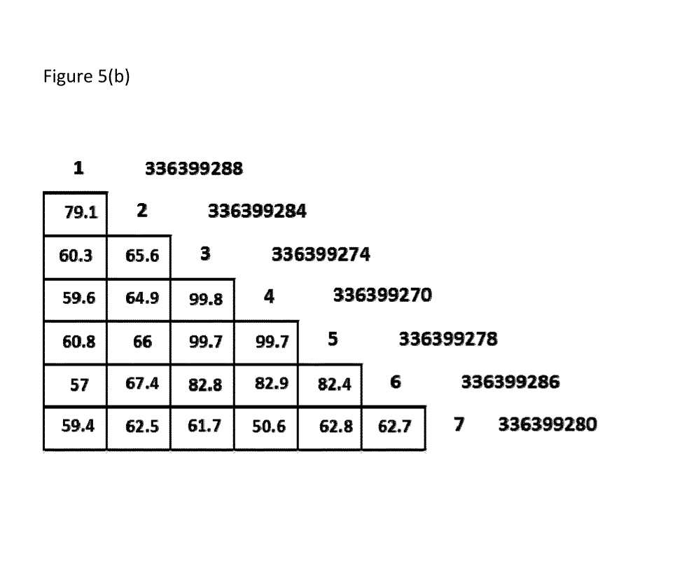

For REP-PCR, frozen pellets of Clostridium pharus and four other Clostridium species (C. ljungdahlii, C. ragsdalei, C. carboxidivorans, C. kluyveri) were sent to an outside lab for analysis by the commercially available Diversilab system. The isolates of Clostridium pharus grouped closely together by this analysis (99.7% similarity) were most similar to C. carboxidivorans with a similarity index range of 82.4-82.9% (FIG. 5(a) and FIG. (b)). The isolates were well separated from C. kluyveri.

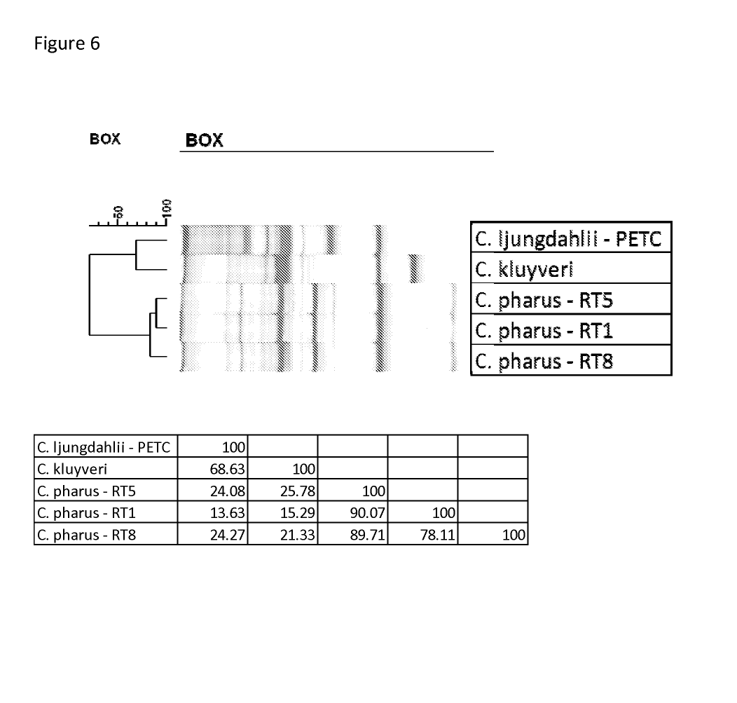

Box-PCR analysis was performed on the three Clostridium pharus isolates as well as C. kluyveri and C. ljungdahlii (FIG. 6). The similarity indices of the Clostridium pharus isolates compared to the two species tested were similar with the analysis showing good separation between the isolates and the test species.

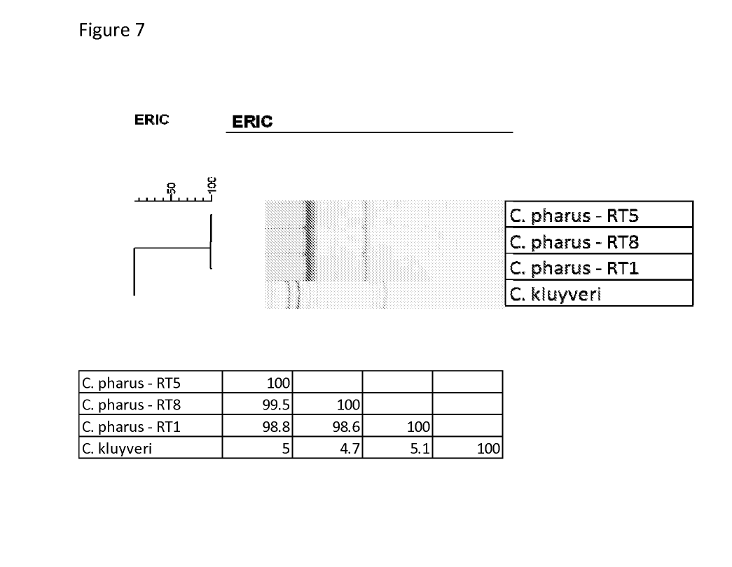

ERIC-PCR analysis was performed on three Clostridium pharus isolates and C. kluyveri (FIG. 7). The Clostridium pharus isolates showed a high degree of similarity to each other and a low degree of similarity to C. kluyveri by this method.

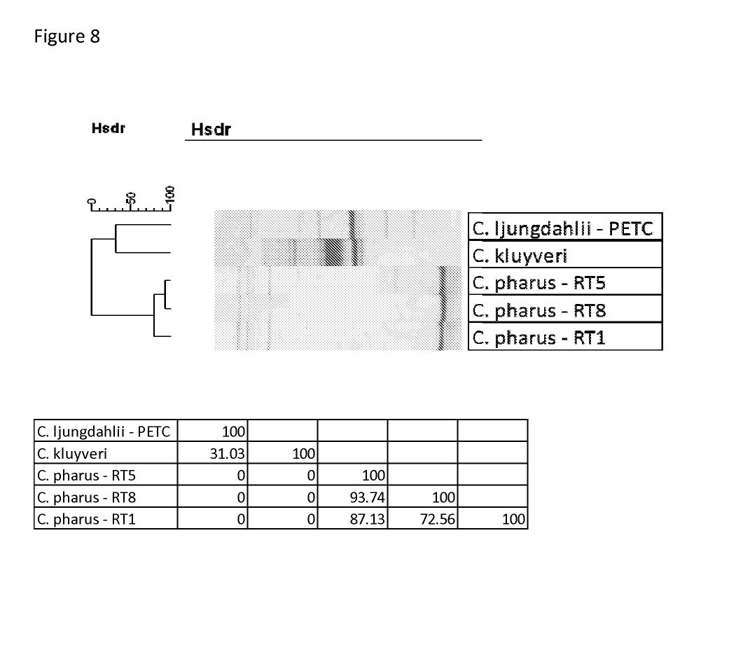

HsdR-PCR analysis was performed on three Clostridium pharus isolates as well as C. kluyveri and C. ljungdahlii (FIG. 8). The similarity indices of the Clostridium pharus isolates had a high degree of similarity to each other with this method and low similarity to the two other species tested.

Clostridium pharus showed a low degree of similarity to the other species tested with all four of the DNA fingerprinting methods performed. All methods performed resulted in a low degree of similarity between Clostridium pharus and C. kluyveri.

### Example 5

### Temperature and pH Optima

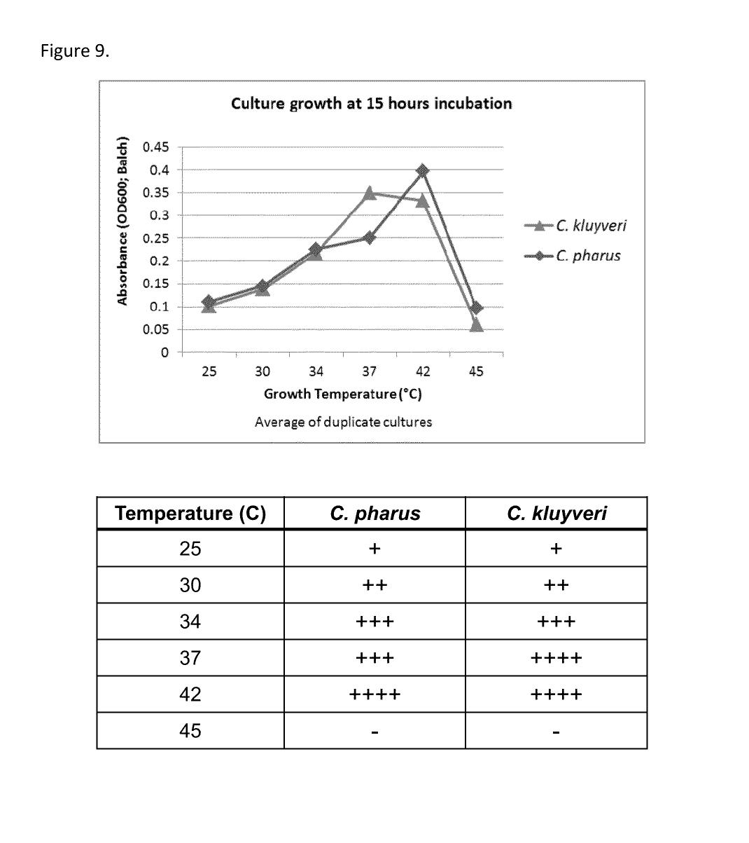

A temperature optima growth experiment was performed in Balch tubes for both Clostridium pharus and C. kluyveri. Tubes were incubated at a range of temperatures without shaking (stationary) and growth was measured periodically by optical density (OD 600; Balch tube readings). The data is presented as the average of duplicate tubes. The temperature range tested was from 25° C. to 45° C. Clostridium pharus showed a temperature optima at 42° C. while C. kluyveri's optimal temperature appeared to be within the 37-42° C. range (FIG. 9). Growth occurred for both species at all temperatures tested except 45° C.

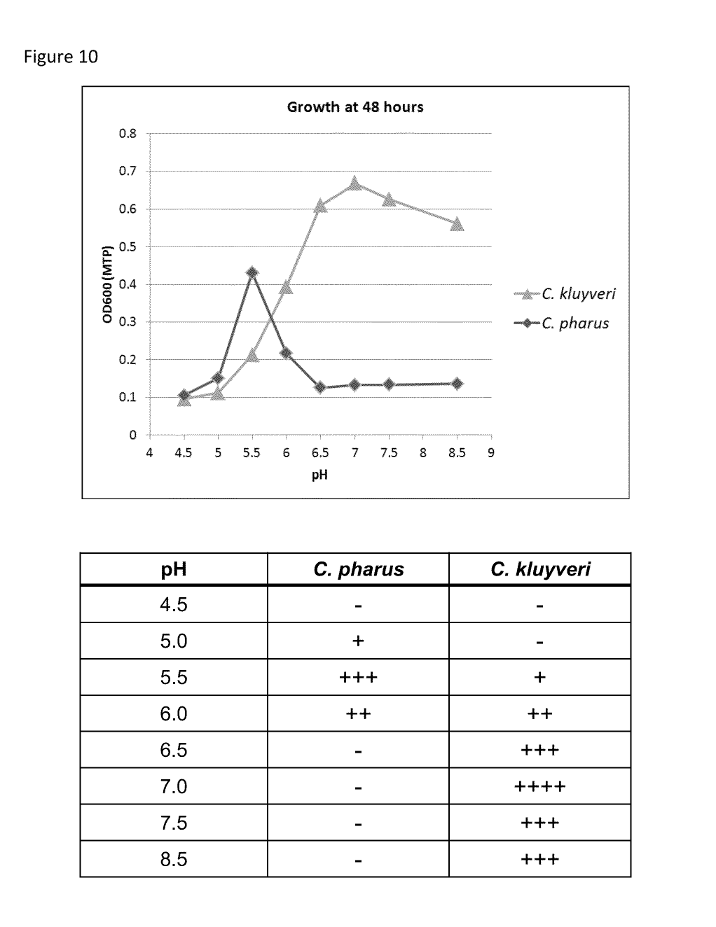

The pH optima for Clostridium pharus and C. kluyveri was determined in a microtiter plate (MTP) growth assay. The range tested was from pH 4.5 to 8.5 and growth was monitored periodically by subsampling the plate wells and reading the optical density on a MTP plate spectrophotometer. Duplicate wells were monitored for each species and pH and the data were the average of the duplicates. Clostridium pharus exhibited a marked pH optimum at pH 5.5 and grew in the range of pH 5.0 to 6.0 (FIG. 10). No growth occurred outside this range within the time period of the experiment. In contrast, C. kluyveri grew best at a pH range of 6.5 to 7.5 and had an overall growth range of 5.5 to 8.5 (FIG. 10).

### Example 6

### Physiology

Potential substrates for growth were determined for Clostridium pharus and C. kluyveri using a commercially available Biolog plate assay. The Biolog system tests for the ability of a culture to use a substrate as an electron donor to reduce a tetrazolium dye. The reduced dye produces a color that is the basis for an absorbance assay to determine a positive test result. As such, this system only indicates the ability of the culture to use the substrate supplied as an electron donor and does not necessarily indicate the ability of the culture to grow with the substrate as carbon or energy source. The plates used were 96-well format and contained 95 different substrates and a water blank.

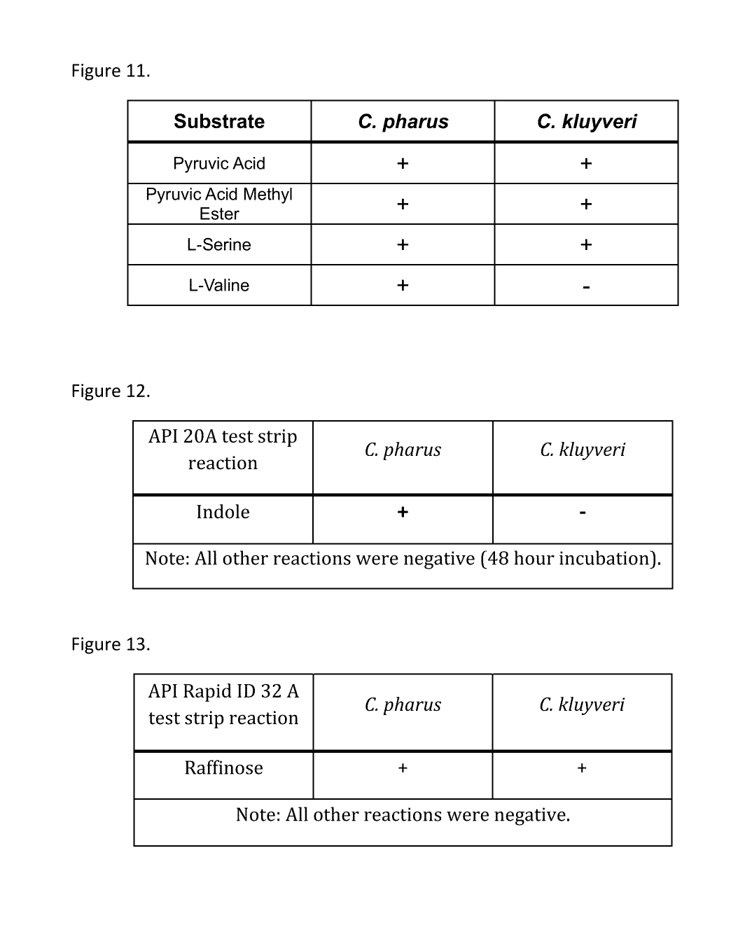

Based on only the positive tests (four positive wells out of 95 for C. pharus), both Clostridium pharus and C. kluyveri were able to use pyruvate, pyruvate methyl ester and L-serine to reduce the dye (FIG. 11). In addition, Clostridium pharus also gave a positive result with L-valine (FIG. 11).

Two additional commercially available strain identification test kit systems were performed on Clostridium pharus and C. kluyveri. The API 20A and API Rapid ID 32A tests (Biomerieux) were performed by inoculating the test strips with previously grown cultures.

Using the API 20 A test, Clostridium pharus returned a positive result for the indole formation test while C. kluyveri was negative for the test (FIG. 12). All other tests in this kit were negative for both species. Both Clostridium pharus and C. kluyveri and C. kluyveri were positive with the raffinose test of the Rapid 32A test strip with all other test wells being negative (FIG. 13).

### Example 7

### Growth with Other Alcohols/Acids

In addition to growth with ethanol and acetate, Clostridium kluyveri is able to grow with propanol and acetate or ethanol and succinate as substrates (Kenealy and Waselefsky 1985). An experiment was performed to determine if Clostridium pharus could also utilize these substrates. The substrates were added at a nominal concentration of 1% for the alcohols and 100 or 50 mM for acetate or succinate, respectively. Growth was performed in Balch tubes and monitored by optical density.

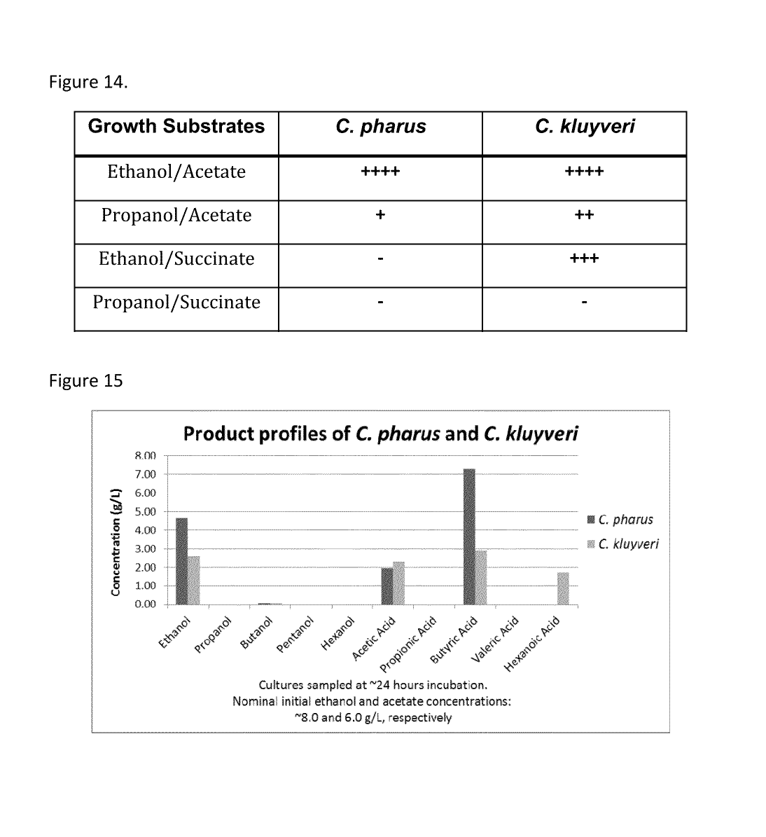

C. kluyveri grew well with ethanol/acetate, propanol/acetate and ethanol/succinate with ethanol/acetate yielding the most rapid growth from inoculation. In addition to ethanol/acetate (FIG. 14) Clostridium pharus was able to grow with propanol/acetate but this growth was slower and resulted in a lower cell density (FIG. 14). Unlike C. kluyveri, Clostridium pharus was unable to grow with succinate as a substrate.

### Example 8

### Product Profile from Ethanol/Acetate Growth

The products of growth on ethanol and acetate for both Clostridium pharus and C. kluyveri were determined after approximately 24-hours of growth by gas chromatography. Both cultures were grown on the same medium and under the same incubation conditions. Subsamples were removed from the bottles by syringe and filtered through a 0.2 μm membrane prior to analysis.

When grown on ethanol and acetate as substrates, C. kluyveri yielded butyric acid and hexanoic acid as products and Clostridium pharus produced butyric acid only (FIG. 15). Clostridium pharus is distinguished from C. kluyveri in that no hexanoic acid is produced from ethanol/acetate growth.

### Example 9

### Fatty Acid Methyl-Ester Analysis

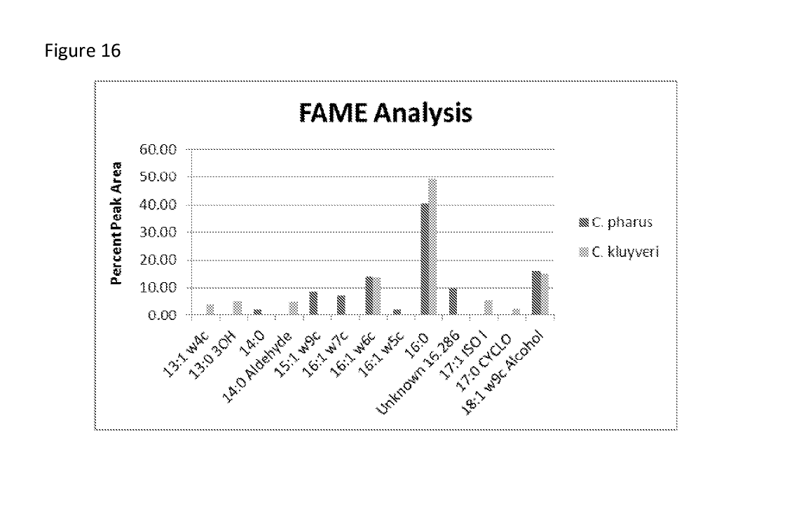

The fatty acid methyl-ester (FAME) profiles of cultures of Clostridium pharus and C. klyveri are shown in FIG. 16. There are differences displayed in the minor components of these profiles. In particular, C. kluyveri exhibits some C-13 and C-17 components that are not present in the C. pharus profile while the C. pharus profile contains a C-15 component as well as an unknown component at an Equivalent Chain Length (ECL) of 16.286 that are not seen in the C. kluyveri profile.

### REFERENCES

(2012). Cobalt and the Naval Air Warfare Center Team Up to Produce a Renewable Jet Fuel From Bio N-Butanol PRNewswire, Cobalt Technologies.
Balch, W. E., G. E. Fox, L. J. Magrum, C. R. Woese and R. S. Wolfe (1979). “Methanogens: reevaluation of a unique biological group.” Microbiol Rev 43 (2): 260-296.
Balch, W. E. and R. S. Wolfe (1976). “New approach to the cultivation of methanogenic bacteria: 2-mercaptoethanesulfonic acid (HS-CoM)-dependent growth of Methanobacterium ruminantium in a pressurized atmosphere.” Appl Environ Microbiol 32 (6): 781-791.
Bredwell, M. D., P. Srivastava and R. M. Worden (1999). “Reactor Design Issues for Synthesis-Gas Fermentations.” Biotechnology Progress 15 (5): 834-844.
Dwidar, M., J. Y. Park, R. J. Mitchell and B. I. Sang (2012). “The future of butyric acid in industry.” ScientificWorldJournal 2012: 471417.
Janda, J. M. and S. L. Abbott (2007). “16S rRNA gene sequencing for bacterial identification in the diagnostic laboratory: pluses, perils, and pitfalls.” J Clin Microbiol 45 (9): 2761-2764.
Kenealy, W. R. and D. M. Waselefsky (1985). “Studies on the substrate range of Clostridium kluyveri; the use of propanol and succinate.” Archives of Microbiology 141: 187-194.
Klasson, K. T., C. M. D. Ackerson, E. C. Clausen and J. L. Gaddy (1992). “Biological conversion of synthesis gas into fuels.” International Journal of Hydrogen Energy 17 (4): 281-288.
Konstantinidis, K. T. and J. M. Tiedje (2005). “Towards a genome-based taxonomy for prokaryotes.” J Bacteriol 187 (18): 6258-6264.
Seedorf, H., W. F. Fricke, B. Veith, H. Bruggemann, H. Liesegang, A. Strittmatter, M. Miethke, W. Buckel, J. Hinderberger, F. Li, C. Hagemeier, R. K. Thauer and G. Gottschalk (2008). “The genome of Clostridium kluyveri, a strict anaerobe with unique metabolic features.” Proc Natl Acad Sci USA 105 (6): 2128-2133.
Stackebrandt, E. and J. Ebers (2006). “Taxonomic parameters revisited: tarnished gold standards.” Microbiology Today (November): 152-155.
Tindall, B. J., R. Rossello-Mora, H. J. Busse, W. Ludwig and P. Kampfer (2010). “Notes on the characterization of prokaryote strains for taxonomic purposes.” Int J Syst Evol Microbiol 60 (Pt 1): 249-266.
Zhang, C., H. Yang, F. Yang and Y. Ma (2009). “Current progress on butyric acid production by fermentation.” Curr Microbiol 59 (6): 656-663.

1. An isolated biologically pure culture of the microorganism Clostridium pharus having the genotypic characteristics of ATCC PTA No. 13419.

2. An isolated biologically pure culture of the microorganism Clostridium pharus, ATCC No. PTA-13419 having the ability, under anaerobic conditions, to produce butyrate from energy sources selected from the group consisting of ethanol, acetate, and propanol, or combinations thereof.

3. (canceled)

4. (canceled)

5. (canceled)

6. An isolated biologically pure culture of the microorganism Clostridium pharus comprising the 16S rDNA sequence as set forth in SEQ ID NO. 1.

7. A method of producing butyrate comprising the steps of contacting the Clostridium pharus with energy sources selected from the group consisting of ethanol, acetate and propanol, or combinations thereof under conditions, which allow the Clostridium pharus to convert the energy sources to butyrate.

8. The method of claim 7 wherein the energy sources are selected from the group consisting of ethanol and acetate, or combinations thereof.

9. The method of claim 7 wherein the energy sources are selected from the group consisting of propanol and acetate, or combinations thereof.


SEQUENCE LISTING (1 sequence)

SEQ ID NO: 1 - DNA, 1432 bp, Clostridium pharus
caccccaattactaaccccaccttcggccgcgtcctcctaagttagactacggacttcgggtgttgccagctctcatggtgtgacgggcggtgtgtacaagacccgggaacgtattcaccgcgacatgctgattcgcgattactagcaactccaacttcatgcaggcgggtttcagcctgcaatccgaactgggaacagtttttgagatttgctcctcctcgcggtattgcttctctctgttctgcccattgtagcacgtgtgttgccctggacataaggggcatgatgatttgacgtcatccccaccttcctccacgttaaccgcggcagtctcgttagagtgcccacctttcgtgctggcaactaacgacaagggttgcgctcgttgcaggacttaacctaacatctcacgacacgagctgacgacaaccatgcaccacctgtctccctgccccggagggcttcctccattacaagttattcaggggatgtcaagtccaggtaaggttcttcgcgttgcttcgaattaaaccacatgctccgctgcttgtgcgggtccccgtcaattcctttgagttttaatcttgcgatcgtacttcccaggcggagtacttattgtgtttactgcggcacagggggggtcgataccccctacacctagtactcatcgtttacggcgtggactaccagggtatctaatcctgtttgctacccacgctttcgtgcctcagcgtcagttacagtccagagaaccgccttcgccactggtgttcttcctaatctctacgcatttcaccgctacactaggaattccgttctcctctcctgcactctagatctccagtttgaaatgcactgcccaagttaagcccgggtatttcacatctcacttaaacatccgcctgcacaccctttacgcccagtaattccggacaacgctcgccacctacgtattaccgcggctgctggcacgtagttagccgtggcttcctcttccggtaccgtcattatcgtcccggatgacagagttttacgatccgaaaaccttcttcactcacgcggcgttgctgcatcagggtttcccccattgtgcaatattccccactgctgcctcccgtaggagtctggaccgtctctcagttccaatgtggccgttcaccctctcaggtcggctacgcatcgttgccttggtgcgcttttaccacaccaactagctaatgcgccgcgggcccatctcaaagcggatttctcctttaaggtatctaccatgcggtttttaccttttatgcggtattaatctccctttcgggaggctatccccctctttgaggcaggttacccacgtgttactcacccgtctgccgctaatccacccccggaggggcttcttcgctcgacttgcatgtgttaggcacgccgccagcgttcgtc